(19) United States
(12) Patent Application Publication
(10) Pub. No.: US 2020/0390807 A1
(43) Pub. Date: Dec. 17, 2020

(54) COMPOSITION AND METHOD FOR TARGETING NATURAL KILLER CELLS IN IMMUNOTHERAPY TO OVERCOME TUMOR SUPPRESSION WITH MANGANESE DIOXIDE NANOPARTICLES

(71) Applicant: UNIVERSITY OF FLORIDA RESEARCH FOUNDATION, INCORPORATED, GAINESVILLE, FL (US)

(72) Inventors: Isaac ADJEI, Gainesville, FL (US); Blanka SHARMA, Gainesville, FL (US)

(21) Appl. No.: 16/971,425

(22) PCT Filed: Feb. 20, 2019
(86) PCT No.: PCT/US2019/018677

§ 371 Date: Aug. 20, 2020

Related U.S. Application Data
(60) Provisional application No. 62/632,922, filed on Feb. 20, 2018.

(51) Int. Cl. A61K 33/32; C07K 14/71; A61K 35/17; A61P 37/04

(52) U.S. Cl. CPC A61K 33/32; C07K 14/71; B82Y 5/00; A61P 37/04; A61K 35/17

(57) ABSTRACT

The present invention provides manganese dioxide nanoparticles complexed with one or more nucleic acid sequences for introduction into NK cells for an immunotherapy treatment. The nanoparticles discussed herein target and silence TGF-β in order to reactivate NK cells that have been inactivated or suppressed. Provided is a method of inducing an anti-cancer immune response in a subject, comprising administering to immune cells of the subject a pharmaceutically effective amount of a nanoparticle composition comprising a manganese dioxide nanoparticle complexed with a nucleic acid sequence in an amount sufficient to induce, enhance, or promote an immune response against the cancer in the subject. The invention also comprises a manganese dioxide nanoparticle formulation, comprising the manganese dioxide nanoparticle(s) as described and a pharmaceutically acceptable carrier. The invention also relates to a method of scavenging reactive oxygen species in a tissue, comprising contacting the tissue with the manganese dioxide nanoparticle(s) as described herein.

### FIELD

The invention relates to the field of bioactive nanoparticles (NPs) tagged with RNA that can scavenge reactive oxygen species to target tumors in a subject in order invoke an immunotherapeutic response to the tumors.

### BACKGROUND

Natural killer cells (NK cells) are a critical component of both the innate and the adaptive immune system. These NK cells rapidly detect and destroy infected or malignant cells. Immune cells typically detect major histocompatibility complex (MHC) presented on infected cell surfaces, triggering cytokine release which causes lysis or apoptosis. NK cells, specifically, recognize stressed cells even in the absence of antibodies and MHC, speeding up the immune reaction. The role of NK cells in the innate and adaptive immune responses, and research using NK cell activity is invaluable as a potential cancer therapy.

NK cells are often suppressed by tumor cells, inhibiting the cytotoxic functions of these cells. Therapies currently include viral vectors used to reactivate NK cells which have become suppressed by a tumor cell. However, viral vectors include safety issues such as the potential for infection of healthy cells, insertion of the new gene in an incorrect location causing a harmful mutation, or an immune reaction caused by the viral vector.

Transforming growth factor-β (TGF-β) is an immunosuppressive cytokine, which can be beneficial in resolving inflammation and preventing autoimmunity. However, TGF-β presents issues with respect to its inhibition of antitumor immune responses by inhibiting, for example, NK cells. TGF-β inhibits activation and function of NK cells by repressing the mTOR pathway in one example.

In addition, there is a need in the art for reduction of oxidative stress generally, because this condition is a causative factor in so many disease conditions.

### SUMMARY

The nanoparticles discussed herein target and silence TGF-β in order to reactivate NK cells that have been inactivated or suppressed. The nanoparticle embodiments discussed herein include a manganese dioxide nanoparticle (MNO₂-NP) complexed with a silencing or expression-interfering molecule. Silencing or expression interfering may occur, for example, by silencing via antisense, miRNA, shRNA, or siRNA, for example. In one embodiment, the nanoparticle may be tagged to an interfering molecule selected from the group consisting of a phosphothioate morpholino oligomer (PMO), miRNA, siRNA, methylated siRNA, treated siRNAs, shRNA, antisense RNA, a dicer-substrate 27-mer duplex, and combinations thereof.

siRNA molecules can be prepared against a portion of a nucleotide sequence encoding transforming growth factor (TGF)-β, and TGF beta receptors I and II, according to the techniques provided in U.S Patent Publication Nos. 20160289315 and 20060110440, incorporated by reference herein, and used as therapeutic compounds. shRNA constructs are typically made from one of three possible methods; (i) annealed complementary oligonucleotides, (ii) promoter based PCR or (iii) primer extension. See Design and cloning strategies for constructing shRNA expression vectors, Glen J McIntyre, Gregory C Fanning BMC Biotechnology 2006, 6:1 (5 Jan. 2006).

For background information on the preparation of miRNA molecules, see e.g., U.S. patent applications 20110020816, 2007/0099196; 2007/0099193; 2007/0009915; 2006/0130176; 2005/0277139; 2005/0075492; and 2004/0053411, the disclosures of which are hereby incorporated by reference herein. See also U.S. Pat. Nos. 7,056,704 and 7,078,196 (preparation of miRNA molecules). Synthetic miRNAs are described in Vatolin, et al, 2006 J Mol Biol 358, 983-6 and Tsuda, et al 2005 Int J Oncol 27, 1299-306. See also patent document WO2011/127202 for further examples of interfering molecules for targeting TGF-β, for example.

The nanoparticles of this invention scavenge reactive oxidative species (hydrogen peroxide) within tumors. Hypoxia is a characteristic feature of locally advanced solid tumors, which results from an imbalance between the intake and consumption of oxygen caused by abnormal vessels in the tumor and the rapid proliferation of tumor cells. Hypoxia is a key marker of tumor progression. Manganese dioxide nanoparticles are effective in localizing to tumors, as a result of favorable physical and chemical properties for tumor accumulation and diffusion and are capable of generating oxygen specifically in sites of tumor hypoxia. This occurs because the manganese dioxide nanoparticles have high reactivity toward hydrogen peroxide (H₂O₂), resulting in oxygen production in hypoxic regions of tumors. In addition, TGF-β is known to suppress NK-cells. The combination of changing the hypoxic state of the tumor microenvironment while silencing TGF-β provides a synergistic effect resulting in activation of NK-cells against the tumor cells.

According to one embodiment, provided is a method of inducing an anti-cancer immune response in a subject, comprising administering to immune cells of the subject a pharmaceutically effective amount of a nanoparticle composition comprising a manganese dioxide nanoparticle (MnO₂-NP) complexed with a nucleic acid sequence in an amount sufficient to induce, enhance, or promote an immune response against the cancer in the subject.

In addition, the invention relates to manganese dioxide nanoparticles that are able to deliver nucleic acids (e.g., siRNA, shRNA, or microRNA) to NK cells in a subject to reactivate the NK cells to overcome tumor suppression. In particular, the invention concerns a manganese dioxide nanoparticle, which is produced by (a) adding poly(allylamine hydrochloride) and KMnO4 in a 1:1 ratio to water with mixing; (b) washing the nanoparticles formed and suspending the nanoparticles in water; (c) adding succinimidyl valerate poly(ethylene glycol) to the nanoparticles, with further mixing; and (d) washing the nanoparticles. The invention further includes tagging a silencing oligonucleotide to the manganese dioxide nanoparticle to form a manganese dioxide nanoparticle-nucleic acid complex. These particles preferably have a size of less than 100 nm, in one non-limiting embodiment.

The invention also comprises a manganese dioxide nanoparticle formulation, comprising the manganese dioxide nanoparticle(s) as described and a pharmaceutically acceptable carrier. For example the invention contemplates a manganese dioxide nanoparticle formulation, comprising (a) a plurality of manganese dioxide nanoparticles having a size by transmission electron microscopy of about 5-30 nm and tagged with a a silencing oligonucleotide to target a TGF-beta; and (b) an aqueous pharmaceutically acceptable carrier.

The invention also relates to a method of inducing an anti-cancer immune response in a subject in need thereof, comprising administering to the subject the manganese dioxide nanoparticle(s) as described herein. The subject can be a mammal, preferably a human. The subject may be diagnosed with a solid tumor, in one non-limiting embodiment. Treatment according to the invention can be by any convenient route of administration, but preferably is by injection or infusion.

The invention also relates to a method of scavenging reactive oxygen species in a tissue, comprising contacting the tissue with the manganese dioxide nanoparticle(s) as described here and to methods of targeting NK cells and reactivating NK cells which have been suppressed by a tumor cell in order to overcome the tumor immune suppression and activate the cytotoxic function of the NK cell. Without being bound to any mechanistic theory, it is believed that the NPs act in two ways:

1. Decrease hypoxia, oxidative stress and acidity of tumor. These properties of the tumor inhibit immune response, not just NK cells.

2. Delivery of an siRNA to TGF, or Transforming growth factor beta receptors 1(TGFβRI) or 2 (TGFβRII) to the NK cells to prevent their response to TGF. Without the receptor, NK cells are not susceptible to the effects of TGFb.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIGS. 1A-1F: Characteristics of NPs. (A) Absorbance spectra of showing reduction KMnO4 to MnO₂. (B) Fourier-transform infrared spectroscopy plot confirming pegylation of MnO₂-NPs (pMnO₂-NP). (C) Stability of pMnO₂-NPs in complete cell culture media (D) Transmission electron microscopy micrograph of pMnO₂-NP (E) Hydrodynamic diameter of pMnO₂-NP (F) Changes in zeta potential of pMnO₂-NP.

FIGS. 2A-2B: siRNA complexation and protection by pMnO2-NPs. (A) Complexation of 100 pmole of siRNA against TGFBR2 to pMnO2-NPs. Complete complexation observed with 5 and 10 μg NP. (B) Protection of siRNA against TGFBR2 by pMnO2-NPs from RNAseI degradation. 100 pmole of siRNA was complexed to 10 μg NP before treatment with RNAseI.

FIGS. 3A-3D: pMnO2-NPs mediate knockdown of TGFBR2 in NK-92 cells. (A) Uptake of pMnO2-NPs by NK-92 cells (B) pMnO2-NPs do not induce apoptosis in NK-92 cells. TGFBR2 knockdown in NK-92 cells evaluated by quantitative real time PCR (C) and immunofluorescent staining (D).

FIG. 4 is a graphical representation of NK cells treated with TGFBR2 siRNA-NPs before TG-beta1 exposure and the ability to kill A549 cells.

FIGS. 5A-5B: NP-mediated knockdown of TGFBR2 protect NK-92 cells from TGF-β. Interferon gamma production by NK cells in response to A549 (A) and H1299 (B) lung cancer cells. *p<0.05, **p<0.01, ****p<0.0001, ns=no statistical significance

FIG. 6: Flow cytometry showing infiltration of NK-92 cells into H1299 lung cancer cell spheroids.

FIG. 7 provides a diagraming showing a synthesis scheme for making MnO2 nanoparticles.

FIG. 8. Tumor accumulation of MnO2 NPs. Fluorescently tagged MnO2 NPs were injected intravenously into A549 tumor bearing NSG mice (left). Control mice were not injected with NPs (right). Tumor accumulation of NPs was observed after 24 hours (left).

### DETAILED DESCRIPTION

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent +variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. Unless defined otherwise, all technical and scientific terms used herein have the same meaning as commonly understood by one of ordinary skill in the art.

A mammal, as used herein, refers to any of the class mammalia, including, but not limited to laboratory, farm, or companion animals such as rats, mice, rabbits, bovines, equines, ovines, porcines, canines, felines, simians, humans, and the like.

A nanoparticle or nanoparticles, as used herein, refers to particles having a size from about 1 nm to about 250 nm, preferably about 5 nm to about 100 nm, and more preferably about 15 nm to about 50 nm.

A manganese dioxide nanoparticle, as used herein, refers to a nanoparticle comprising manganese dioxide, preferably containing at least about 33% manganese dioxide, more preferably about 33% to 66% manganese dioxide, and most preferably about 50% manganese dioxide. Regarding the use of the term “about” with respect to describing composition, size or charge of the nanoparticles, “about” includes the stated value and values up to 15%, 20%, or 25% lesser or greater than the stated value.

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “co-administered, “co-administering,” or “concurrent administration”, when used, for example with respect to administration of an exemplary therapeutic agent with another exemplary therapeutic agent, or a conjunctive agent along with administration of an exemplary therapeutic agent refers to administration of the exemplary therapeutic agent and the other exemplary therapeutic agent and/or conjunctive agent such that both can simultaneously achieve a physiological effect. The two agents, however, need not be administered together. In certain embodiments, administration of one agent can precede administration of the other, however, such co-administering typically results in both agents being simultaneously present in the body (e.g. in the plasma) of the subject.

The term “cancer” or “tumor” as used herein means is intended to include any neoplastic growth in a patient, including an initial tumor and any metastases. The cancer can be of the liquid or solid tumor type. Liquid tumors include tumors of hematological origin (hematological cancer), including, e.g., myelomas (e.g., multiple myeloma), leukemias (e.g., Waldenstrom's syndrome, chronic lymphocytic leukemia, other leukemias), and lymphomas (e g, B-cell lymphomas, non-Hodgkins lymphoma). Solid tumors can originate in organs, and include cancers such as lung, breast, prostate, ovary, colon, kidney, and liver. In a non-limiting embodiment, cancer pertains to solid tumors.

The term “cancerous cell” or “cancer cell” as used herein means a cell that shows aberrant cell growth, such as increased cell growth. A cancerous cell may be a hyperplastic cell, a cell that shows a lack of contact inhibition of growth in vitro, a tumor cell that is incapable of metastasis in vivo, or a metastatic cell that is capable of metastasis in vivo. Cancer cells include, but are not limited to, carcinomas, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large B cell lymphoma, malignant lymphoma, plasmocytoma, reticulum cell sarcoma, or Hodgkins disease).

The term “complexed nanoparticles” as used herein refers to manganese dioxide nanoparticles to which a nucleic acid sequence (e.g., inhibitory oligonucleotide) has been bound, whether with or without a linking molecule.

The terms “subject,” “individual,” “host,” and “patient,” are used interchangeably herein to refer to an animal being treated with one or more enumerated agents as taught herein, including, but not limited to, simians, humans, avians, felines, canines, equines, rodents, bovines, porcines, ovines, caprines, mammalian farm animals, mammalian sport animals, and mammalian pets. A suitable subject for the invention can be any animal, preferably a human, that is suspected of having, has been diagnosed as having, or is at risk of developing a disease that can be ameliorated, treated or prevented by administration of one or more enumerated agents.

The term “TGF related expression” pertains to expression of TGF-beta (beta 1a or 1b) and TGF-beta receptor (beta 1 receptor or beta 2 receptor).

The term “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a disease, disorder, or condition or one or more symptoms or manifestations of a disease, disorder or condition.

A “therapeutically effective amount” refers to an amount which, when administered in a proper dosing regimen, is sufficient to reduce or ameliorate the severity, duration, or progression of the disorder being treated (e.g., cancer), prevent the advancement of the disorder being treated (e.g., cancer), cause the regression of the disorder being treated (e.g., cancer), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

Oxidative stress, as used herein, refers to the systemic manifestation of too high a level of reactive oxygen species in a biological system or animal body, organ or tissue, such that the natural system is not capable of removing or neutralizing the reactive oxygen species or repairing the damage they can cause, resulting in impaired performance of cells.

### 2. Overview

Certain tumors are able to develop an immunosuppressive microenvironment in order to evade detection and/or treatment. This microenvironment inhibits the cytotoxic functions of NK cells, wherein the NK cells are inactivated. Systems and methods of the inventive embodiments can target NK cells, and activate or reactivate their tumor-destroying capabilities by introducing genetic material into NK cells. This genetic material, i.e. nucleic acids (RNA and DNA molecules) or drugs may be delivered to NK cells to reactivate its cancer killing function. In some non-limiting embodiments, a silencing RNA may be delivered to NK cells.

### 3. Description of Exemplary Embodiments

Manganese dioxide nanoparticles preferably are produced and formulated in aqueous solution and stabilized with polyethylene glycol succinimidyl valerate. Preferred methods for producing the formulations are given in Example 1 below.

Nanoparticles known in the art generally are about 1-100 nm in diameter and can be found in different shapes. The nanoparticles of this invention, however, are generally spherical and preferably are about 5 nm to about 100 nm, and most preferably about 5 nm to about 50 nm, as determined by transmission microscopy, or about 15 nm to about 200 nm as determined by dynamic light scattering.

Hydrogen peroxide decomposes naturally at a very slow rate. Manganese dioxide in the nanoparticles catalyzes the release of oxygen from hydrogen peroxide according to the reaction:

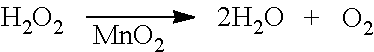

The oxygen escapes as a gas, leaving water, with the reaction proceeding at a much faster rate. The manganese dioxide, acting as a catalyst, remains unchanged.

The nanoparticles according to the invention preferably are formulated to form a pharmaceutical composition using a pharmaceutically acceptable carrier or excipient for administration to a subject. The terms “pharmaceutically acceptable carrier,” “pharmaceutically acceptable excipient,” or “pharmaceutically acceptable vehicle” refer to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art and are well known in the art. Any of the compounds and compositions described in “Remington: The Science and Practice of Pharmacy” (20th edition, Gennaro (ed.) and Gennaro, Lippincott, Williams & Wilkins, 2000, are contemplated for use with the compositions of the invention.

Preferably, the nanoparticles of the invention are administered as a composition containing a liquid carrier or vehicle for injection. Any liquid carrier that is compatible with the nanoparticles and the body of the subject to which it is intended to be administered may be used. Preferably, the carrier is aqueous, such as water, distilled water, deionized water, saline, buffered saline (e.g., phosphate buffered saline), Ringer's solution or lactated Ringer's solution, with the formulation taking the form of a solution or suspension. In addition, the formulation can be an oil-in-water or water-in-oil emulsion or the like. The formulation also can be provided as a solid for dilution with a liquid carrier prior to administration.

The pharmaceutically acceptable carrier also can contain additional compounds such as pH adjusters (acid or base), solubilizers, emulsifiers, salts, preservatives, antimicrobial compounds, and the like. Acceptable salts can include, but are not limited to acetate, adipate, alginate, ammonium, aspartate, benzoate, benzenesulfonate (besylate), bicarbonate, bisulfate, butyrate, citrate, camphorate, camphorsulfonate, carbonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, magnesium, maleate, malonate, methanesulfonate (mesylate), 2-naphthalenesulfonate, nicotinate, nitrate, oxalate, palmoate, pectinate, persulfate, 3-phenylpropionate, phosphate, picrate, pivalate, potassium, propionate, salicylate, sodium, succinate, sulfate, tartrate, thiocyanate, toluenesulfonate (tosylate) and undecanoate salts, for example.

Oxidative stress refers to a condition in which reactive oxygen species (usually from oxidative metabolism) exist in amounts too high for the biological system in which they are present to detoxify the reactive oxygen species or the reactive intermediates, or to repair the damage caused by the reactive oxygen species. Most of the damage is caused by superoxide radicals, hydroxyl radicals or hydrogen peroxide. Production of peroxides and free radicals can damage proteins, lipids, and DNA, and disrupt normal cellular mechanisms, and may often result in tumor growth, or may be used by tumors to evade the immune system. For example, tumor associated macrophages residing at the site of hypoxic region of tumors have been known to cooperate with tumor cells and promote proliferation and chemo resistance.

The particles according to the invention are able to target NK cells to deliver nucleic acids to those cells and reactivate their tumor-destroying function, and to do so without the need for magnetic stimulation or electroporation, in some nonlimiting embodiments. In one particular nonlimiting embodiment, the nanoparticles may be 150 nm or smaller to facilitate uptake by the NK cells. In another non-limiting embodiment, the nanoparticles may be 100 nm or smaller to facilitate uptake by the NK cells.

In certain embodiments, the complexed manganese dioxide nanoparticles disclosed herein are formulated in a pharmaceutically acceptable carrier under sterile conditions and injected into a subject. The dosage given is easily ascertainable by the person of skill and will depend upon the age of the subject, the locations and number of tumors in the subject, the severity of the condition and other physical parameters unique to the subject being treated. The treatment can be administered daily, weekly or monthly, and can be administered one time or as a series of treatments. The treatment can be given in combination with other pharmaceutical agents in one formulation or in separate formulations to be administered by injection, intravenously or orally.

### Inhibitory Oligonucleotides

Agents that reduce TGF-related expression include isolated small hairpin RNA (shRNA), small interfering RNA (siRNA), antisense RNA, antisense DNA, chimeric antisense DNA/RNA, microRNA, and ribozymes that are sufficiently complementary to specifically bind to a gene or mRNA encoding either TGF, to reduce expression. A significant reduction in TGF-related expression is a reduction of about 50% or more. Agents that reduce TGF-related expression include isolated small hairpin RNA (shRNA), small interfering RNA (siRNA), antisense RNA, antisense DNA, chimeric antisense DNA/RNA, microRNA, and ribozymes that are sufficiently complementary to TGF-beta or TGF-beta receptor specifically bind to a gene or mRNA encoding involved in TGF related expression to reduce such expression.

The complexed nanoparticles disclosed herein may by complexed with inhibitory oligonucleotides including but not limited to antisense nucleic acids or small interfering RNA (siRNA) or shRNA to reduce or inhibit expression and hence the biological activity of TGF related expression. Based on these known sequences of these proteins and genes encoding them, antisense DNA or RNA that are sufficiently complementary to the respective gene or mRNA to turn off or reduce expression can be readily designed and engineered, using methods known in the art. In a specific embodiment of the invention, antisense or siRNA molecules for use in the present invention are those that bind under stringent conditions to the targeted mRNA or targeted gene identified by the Genbank numbers, or to variants or fragments that are substantially homologous to the mRNA or gene encoding TGF-beta or TGF-beta receptor. Examples of these sequences is provided below.

Methods of making antisense nucleic acids are well known in the art. As used herein, the terms “target nucleic acid” encompass DNA encoding the target proteins and RNA (including pre-mRNA and mRNA) transcribed from such DNA. The specific hybridization of a nucleic acid oligomeric compound with its target nucleic acid interferes with the normal function of the target nucleic acid. This modulation of function of a target nucleic acid by compounds which specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA. The overall effect of such interference with target nucleic acid function is modulating or reducing the expression of the protein encoded by the DNA or RNA. In the context of the present invention, “modulation” means reducing or inhibiting in the expression of the gene or mRNA for one or more of the targeted proteins.

The targeting process includes determination of a site or sites within the target DNA or RNA encoding the targeted protein for the antisense interaction to occur such that the desired inhibitory effect is achieved. Within the context of the present invention, a preferred intragenic site is the region encompassing the translation initiation or termination codon of the open reading frame (ORF) of the mRNA for the targeted proteins. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene. Routine experimentation will determine the optimal sequence of the antisense or siRNA.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen which are sufficiently complementary to the target; meaning that the nucleic acids will hybridize sufficiently well and with sufficient specificity, to give the desired effect of inhibiting gene expression and transcription or mRNA translation.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of function, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

While antisense nucleic acids are a preferred form of antisense compound, the present invention comprehends other oligomeric antisense compounds, including but not limited to oligonucleotide mimetics. The antisense compounds in accordance with this invention preferably comprise from about 8 to about 50 nucleobases (i.e., from about 8 to about 50 linked nucleosides). Particularly preferred antisense compounds are antisense nucleic acids comprising from about 12 to about 30 nucleobases. Antisense compounds include ribozymes, external guide sequence (EGS) nucleic acids (oligozymes), and other short catalytic RNAs or catalytic nucleic acids which hybridize to the target nucleic acid and modulate its expression. Nucleic acids in the context of this invention include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Antisense nucleic acids have been employed as therapeutic moieties in the treatment of disease states in animals and man. Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans, for example to down-regulate TGF related expression.

US Patent Application 2004/0023390 (the entire contents of which are hereby incorporated by reference as if fully set forth herein) teaches that double-stranded RNA (dsRNA) can induce sequence-specific posttranscriptional gene silencing in many organisms by a process known as RNA interference (RNAi). However, in mammalian cells, dsRNA that is 30 base pairs or longer can induce sequence-nonspecific responses that trigger a shut-down of protein synthesis and even cell death through apoptosis. Recent work shows that RNA fragments are the sequence-specific mediators of RNAi (Elbashir et al., 2001). Interference of gene expression by these small interfering RNA (siRNA) is now recognized as a naturally occurring strategy for silencing genes in C. elegans, Drosophila, plants, and in mouse embryonic stem cells, oocytes and early embryos (Cogoni et al., 1994; Baulcombe, 1996; Kennerdell, 1998; Timmons, 1998; Waterhouse et al., 1998; Wianny and Zernicka-Goetz, 2000; Yang et al., 2001; Svoboda et al., 2000).

In mammalian cell culture, a siRNA-mediated reduction in gene expression has been accomplished by transfecting cells with synthetic RNA nucleic acids (Caplan et al., 2001; Elbashir et al., 2001). The 2004/0023390 application, the entire contents of which are hereby incorporated by reference as if fully set forth herein, provides exemplary methods using a viral vector containing an expression cassette containing a pol II promoter operably-linked to a nucleic acid sequence encoding a small interfering RNA molecule (siRNA) targeted against a gene of interest.

As used herein RNAi is the process of RNA interference. A typical mRNA produces approximately 5,000 copies of a protein. RNAi is a process that interferes with or significantly reduces the number of protein copies made by an mRNA. For example, a double-stranded short interfering RNA (siRNA) molecule is engineered to complement and match the protein-encoding nucleotide sequence of the target mRNA to be interfered with. Following intracellular delivery, the siRNA molecule associates with an RNA-induced silencing complex (RISC). The siRNA-associated RISC binds the target through a base-pairing interaction and degrades it. The RISC remains capable of degrading additional copies of the targeted mRNA. Other forms of RNA can be used such as short hairpin RNA and longer RNA molecules. Longer molecules cause cell death, for example by instigating apoptosis and inducing an interferon response. Cell death was the major hurdle to achieving RNAi in mammals because dsRNAs longer than 30 nucleotides activated defense mechanisms that resulted in non-specific degradation of RNA transcripts and a general shutdown of the host cell. Using from about 19 to about 29 nucleotide siRNAs to mediate gene-specific suppression in mammalian cells has apparently overcome this obstacle. These siRNAs are long enough to cause gene suppression.

Certain embodiments of the invention are directed to the use of shRNA, antisense or siRNA to block expression of the targeted protein or orthologs, analogs and variants thereof in an animal. The antisense compounds of the invention are synthesized in vitro and do not include antisense compositions of biological origin, or genetic vector constructs designed to direct the in vivo synthesis of antisense molecules.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

The disclosure also contemplates complexing manganese dioxide nanoparticles with ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave targeted mRNA transcripts thereby inhibiting translation. A ribozyme having specificity for a targeted-encoding nucleic acid can be designed based upon the nucleotide sequence of its cDNA. For example, a derivative of a Tetrahymena L-19 IVS RNA can be constructed in which the nucleotide sequence of the active site is complementary to the nucleotide sequence to be cleaved in the targeted mRNA. See, e.g., Cech et al. U.S. Pat. No. 4,987,071; and Cech et al. U.S. Pat. No. 5,116,742.

siRNA molecules that reduce expression of TGF-beta or TGF-beta receptor include, but are not limited to, those taught in US Patent Pub 20130011397. Other siRNA molecules includes those commercially available (e.g. Thermofisher Scientific Catalog Nos AM16708, AM51331, 4392420, and 4390824).

shRNA molecules targeting TGF related expression include, but are not limited to, those taught in US Patent Pub 2018036099 and US20160158210, and commercially available from Sigma Aldrich (for example, product nos SHCLNG-NM_000660).

### 4. Examples

It is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### Materials and Methods

### Nanoparticle Synthesis

MnO₂ NPs were synthesized by reduction of 60 mg potassium permanganate (KMnO4; Acros Organics, Geel, Belgium) in 18 mL ultrapure water with 60 mg poly(allylamine hydrochloride) (PAH; Alfa Aesar, Ward Hill, Mass., USA) in 2 mL of ultrapure water for 30 minutes. After the reaction, the NPs were recovered by centrifugation in Amicon Ultra-15 Centrifugal tubes (Mw cutoff: 100000 Dalton, Millipore Sigma) at 3500 rpm and washed twice with ultrapure water to remove unreacted KMnO4 and PAH. These NPs were reacted with an equivalent weight of acrylate-polyethylene glycol succinimidyl valerate (PEG-SVA) (Mw=3400 D; Laysan Bio Inc., Arab, Ala., USA) for 2 hours to form PEG-MnO₂ NPs (pMnO₂-NP). Alexa Fluor™ 488 tagged pMnO₂-NPs were synthesized by incubating NPs with Alexa Fluor™ 488 Succinimidyl Ester (Life Technologies, Carlsbad, Calif., USA) for 2 hours in the presence of 1-ethyl-3-(3-dimethylaminopropyl)carbodiimide hydrochloride).

### NP Characterization

Pegylation of MnO₂-NPs was confirmed by Fourier-transform infrared spectroscopy (FTIR) (Perkin Elmer Frontier, Perkin Elmer, Waltham, Mass., USA). Colloidal stability of MnO₂ NPs in cell culture media after pegylation was tested at a concentration of 1 mg/mL. The hydrodynamic size and zeta potential of pMnO₂-NP in ultrapure water (200 μg/mL) was determined by Dynamic Light Scattering (NICOMP 3000 ZLS; Particle Sizing Systems, Port Richey, Fla., USA). The core size and distribution of pMnO₂-NPs was confirmed by transmission electron microscopy (TEM) (Tecnai™ FEI Spirit TEM 120 kV, ThermoFisher Scientific, Waltham, Mass., USA).

siRNA Binding and Protection Assay

pMnO₂-NP complexation and protection of siRNA were determined by gel electrophoresis. For complexation, 100 pmol siRNA (Sense: GGACAUCUUCUCAGACAUCtt; Antisense: GAUGUCUGAGAAGAUGUCCtt; Product information: Catalog number: AM51331; Assay ID: 387; ThermoFisher Scientific) was incubated with 0, 2.5, 5 and 10 μg pMnO₂-NP for 15 minutes in a total volume of 10 μL. After incubation, 2 μL of ethidium bromide solution and loading buffer was added to each complexation reaction and run on a 4% agarose gel for 2 hours at 100 V and imaged with a BioRad gel imager.

For the protection experiment, 100 pmol siRNA was incubated with 10 μg pMnO₂-NP for 15 minutes. After complexation, 100 U RNAseI (Ambion, Foster City, Calif.) in 2 μL of 10× phosphate buffered saline (PBS) was added to samples for incubated for 1 hour at 37° C. The RNAseI was inactivated with 0.1% sodium dodecyl sulfate and siRNA desorbed from NPs with heparin. The displaced siRNA was stained with ethidium bromide and run on a 4% agarose gel as above.

### Cell Culture

All cell lines were purchased from American Type Culture Collection (Manassas, Va., USA). The human derived non-small cell lung cancer (NSCLC) cell lines H1299 and A549 were cultured in RPMI 1640 supplemented with 10% (v/v) heat-inactivated fetal bovine serum (FBS), 1% (v/v) penicillin-streptomycin, and 1% (v/v) L-glutamine. The NK-92 cells were cultured in RPMI 1640 without L-glutamine, supplemented with 20% (v/v) heat-inactivated FBS, 1% (v/v) of penicillin-streptomycin, 1% (v/v) L-glutamine, 1% (v/v) MEM non-essential amino acids (NEAA) solution, 1% (v/v) sodium pyruvate and 100 units/mL of IL-2. Cells were maintained at 37° C. and 5% CO₂.

### Cell Uptake and Cytotoxicity Studies

NK-92 cells at a density of 1,000,000 cells in 5 mL uptake media were incubated with 10 μg Alexa Fluor 488 labeled pMnO2-NP for 24 hours. The uptake media consisted of OptiMEM supplemented with 1% insulin transferrin selenium (ITS) and 100 U of IL2. The cells were washed 3× with PBS, stained with Hoescht 33258 dye and imaged.

Cytotoxicity of pMnO2-NPs to NK-92 cells was determined after incubating cells with unlabeled pMnO2-NPs for 24 hours. After washing cells with PBS three times, they were stained with Annexin V following the manufacturer's protocol and apoptotic cells detected by flow cytometry.

### TGFBR2 Knockdown Studies

The siRNA against TGFBR2 (100 pmole) was complexed to 10 μg pMnO2-NP for 15 minutes. After complexation, the NP/siRNA was added to 1,000,000 NK-92 cells in 5 mL uptake media. After 24 hours of incubation, the media with NP/siRNA was replaced with complete NK cell media and incubated for another 24 hours. The knockdown of TGFBR2 was analyzed by immunohistochemistry and quantitative real-time polymerase reaction and compared to cells incubated with 100 pmol siRNA alone or untreated controls.

### Immunofluorescent Staining for TGFBR2

After treatment to knockdown TGFBR2, the NK-92 cells were fixed with 4% paraformaldehyde (PFA) and rinsed with PBS. The cells were permeated with 0.1% (v/v) Triton X-100 in PBS (PBST) for 30 minutes blocked with 1% goat serum in PBST for 1 hour. Cells were then incubated with antibody against TGFBR2 (clone: 16H2L4, Invitrogen, Rabbit monoclonal to human; ThermoFisher Scientific, Waltham, Mass.) overnight at 4° C. The cells were washed 3 times with PBST and incubated with FITC tagged donkey anti-rabbit secondary antibody (Santa Cruz Biotechnology, Dallas, Tex.) at room temperature. Cells were counter-stained with Hoescht 33258 dye for 15 minutes, washed in PBS and analyzed with confocal microscopy.

### Quantitative Real-Time Polymerase Chain Reaction

RNA was extracted from cells 48 hours after incubation with siRNA to TGFBR2 only or NP/siRNA using RNeasy Mini Kit (Qiagen, Germantown, Md.) and converted to cDNA (iScript cDNA Synthesis kit; BioRad, Hercules, Calif.). Real-time PCR (QuantStudio 6 Flex Real-Time PCR System, Applied Biosystems, Foster City, Calif.) TGFBR2 was performed with Fast SYBR™ green master mix (ThermoFisher Scientific, Waltham, Mass.). Gene expression was analyzed using the ΔΔCT method with GAPDH as a housekeeping gene. Gene expression for all groups was compared to untreated control. The primer sequences are as follows:

TGFBR2
(F 5′-ACGTGTTGAGAGATCGAGG-3′/
R 5′-CCCAGCACTCAGTCAACGTC-3′);
GAPDH
(F 5′-TAGACGGGAAGCTCACTGGC-3′/
R 5′-AGGTCCACCACCCTGTTGCT-3′).

### Killing Assay

The cancer cell lines H1299 and A549 (5000 cells) were incubated with 25000 NK cells in a total volume of 200 μL optiMEM media supplemented with 100 U of IL2. After 6 hours of incubation, the media was collected and frozen immediately at −80° C. until use. NK cell killing of cancer cells was quantified by lactose dehydrogenase assay following the manufacturer's instruction. The release of IFN-γ by activated NK cells was measured by ELISA (ThermoFisher Scientific, Waltham, Mass.) following the manufacturer's protocol.

### Cancer Cell Spheroid Formation

Spheroids were formed in 1% agarose coated 96-well plates. To each well in a 96-well plate, 5,000 1,1′-Dioctadecyl-3,3,3′,3′-Tetramethylindocarbocyanine Perchlorate (DiI; ThermoFisher Scientific, Waltham, Mass.) stained H1299 cancer cells in 200 μL complete cancer cell media was added. Plates with cells were centrifuged at 1500 rpm for 10 minutes and kept in an incubator without any disturbance. Spheroids formed within 48 hours and 100 μL of media replaced after 72 hours.

NK-92 Infiltration into Cancer Spheroids

At 96 hour after H1299 spheroid initiation, 25,000 NK-92 cells stained with 3,3′-Dioctadecyloxacarbocyanine Perchlorate (DiO; ThermoFisher Scientific, Waltham, Mass.) were added to each spheroid and incubated for 6 hours. After incubation, the spheroids were carefully rinsed with PBS and fixed with 4% PFA. Spheroids were imaged by confocal imaging to evaluate NK cell infiltration.

### Confocal Imaging

After incubation, cells were washed with PBS and imaged with a Zeiss LSM 710 confocal microscope in scanning mode. The microscope has a diode (405 nm), Argon (458 nm, 488 nm, 518 nm), diode-pumped solid state (561 nm) and HeNe (633 nm) laser modules (Oberkochen, Germany). Images were acquired using the ZEN imaging software (Zeiss, Oberkochen, Germany).

### Statistics

All statistical analysis was performed with GraphPad PRISM 7.01 (La Jolla, Calif.). Error bars indicate standard deviations. Statistical comparison was determined by 1-way ANOVA with Tukey's multiple comparisons tests with outcomes denoted as *p<0.05, **p<0.01, ***p<0.001, ****p<0.0001, ns=no statistical significance.

### Example 1. Manganese Dioxide Nanoparticle Formulation

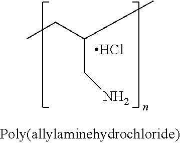

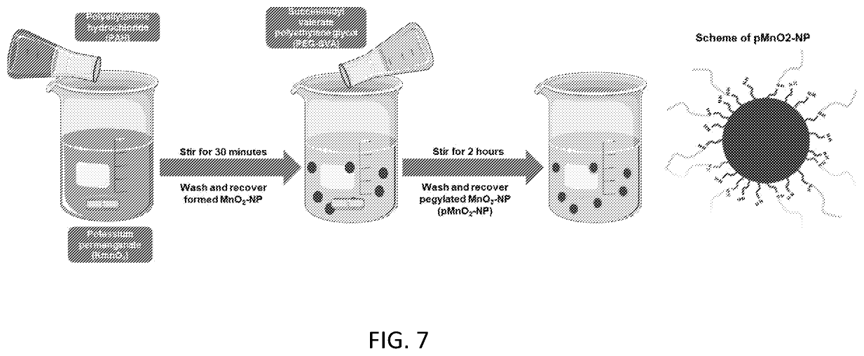

As described above, poly(allylaminehydrochloride) and KMnO4, in a 1:1 ratio, were added to water and stirred for 30 minutes. The resulting particles were washed twice and placed in suspension in water. Succinimidyl valerate poly(ethylene glycol) (PEG-SVA, MW 2000 amu) was added and the mixture stirred for 2 hours. The particles then were washed twice, to produce stabilized manganese dioxide nanoparticles. A synthesis scheme for making the MnO2 nanoparticles is provided in FIG. 7.

Once the particles are formed, an inhibitory oligonucleotide may be tagged to the particles. The inhibitory oligonucleotide may be constructed to target a specific sequence to reactivate NK cells. In one non-limiting example, the oligonucleotides may be used to target and silence TGF-beta in the cell, which is known to inhibit NK cells, and suppress immune response to tumor cells.

Characteristics of pMnO₂-NP

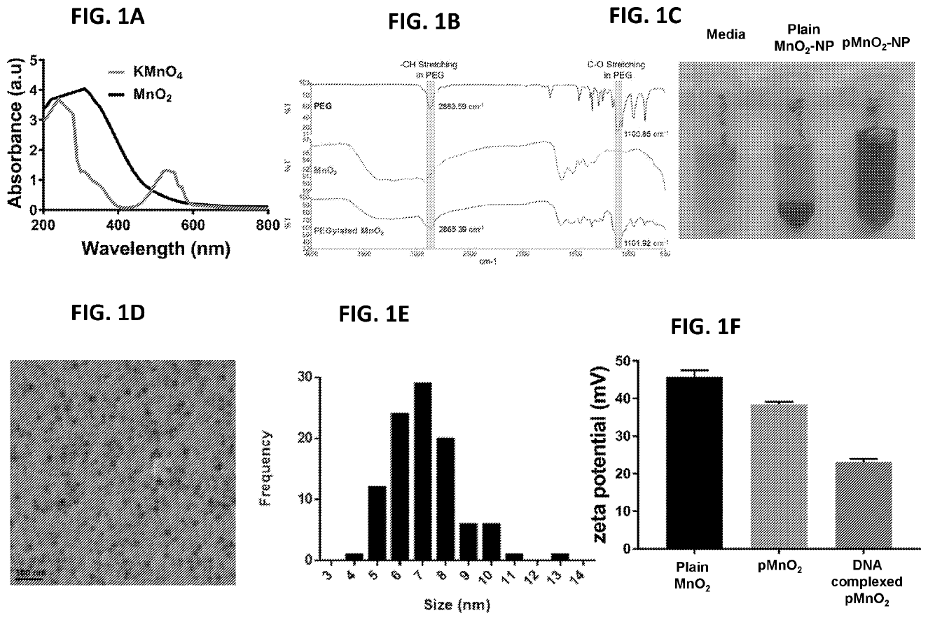

Reduction of KMnO4 by PAH was confirmed by absorbance spectrum analysis (FIG. 1A). The formed MnO₂-NPs did not exhibit colloidal stability in cell culture media. Conjugation of PEG to the NPs (pMnO₂-NP) prevented aggregation (FIG. 1C). Pegylation of NPs was confirmed FTIR which showed the presence of the Carbon-Oxygen bond attributed to PEG on pMnO₂-NP (FIG. 1B). The pegylation did not change the size of pMnO₂ which showed a hydrodynamic size of 15 nm and TEM size of 8 nm (FIG. 1D, E). The pegylation of the NPs decreased their zeta potential to +35 mV and was further decreased to +24 mV after complexation to nucleic acids (FIG. 1F).

### Example 2. Manganese Dioxide Nanoparticles Complexation with Nucleic Acids

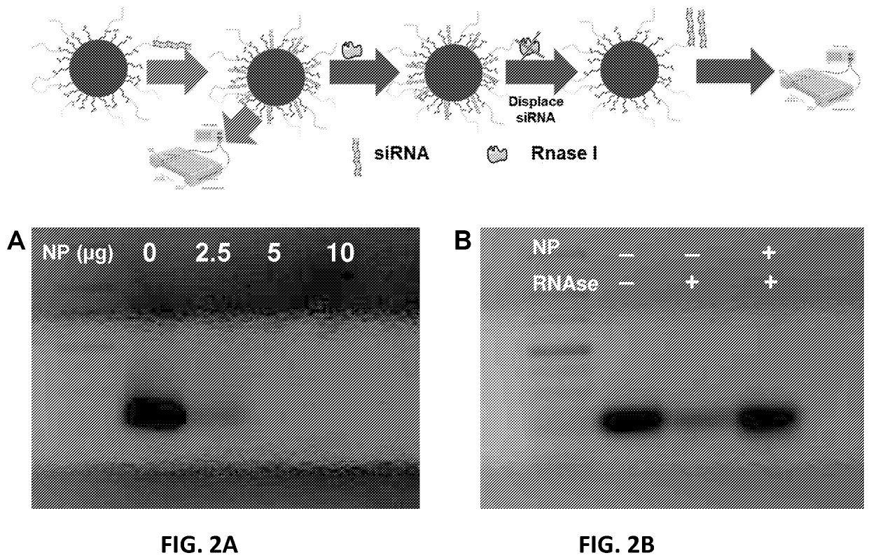

Manganese dioxide nanoparticles were complexed with RNA (Thermo-Fisher, Cat. No. AM51331), and subjected to gel electrophoresis. Results are shown in FIGS. 2A-2B. The effectiveness of pMnO₂-NP complexation to siRNA against TGFBR2 was dependent on the amount of NP used. Incubation of 100 pmole siRNA with 2.5 μg NP resulted in incomplete complexation which was ameliorated by increasing the amount of NP. Using 5- and 10 μg pMnO₂-NPs resulted in complete siRNA complexation (FIG. 2A). Significantly, when the siRNA is complexed to 10 μg pMnO2-NP, they are protected from RNAseI degradation while the uncomplexed siRNA is degraded (FIG. 2B).

The zeta potential of the nanoparticles complexed with nucleic acids becomes less positive and typically have a zeta potential of about +20 mV to about +25 mV.

### Example 3. MnO₂ Uptake by Natural Killer (NK) Cells

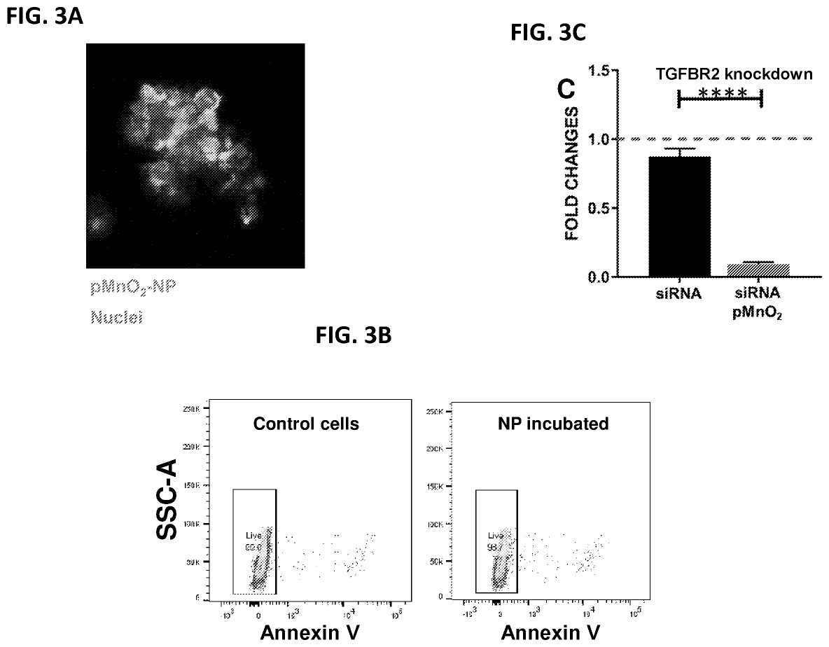

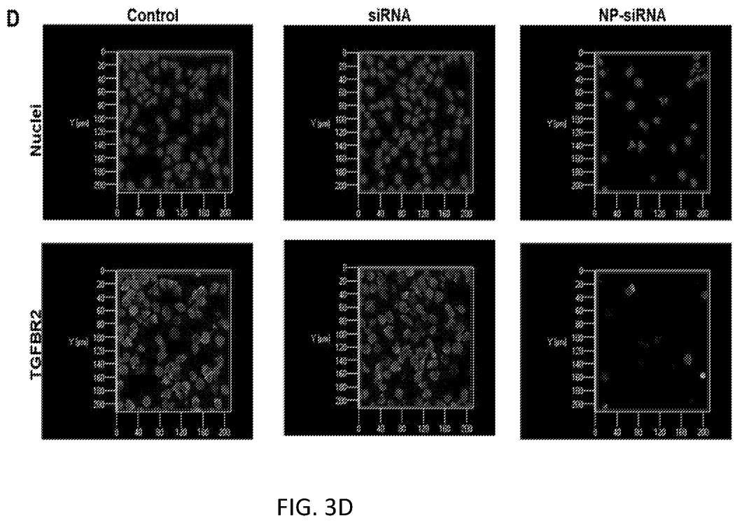

The NK-92 cells endocytosed pMnO₂-NPs without affecting their viability. Fluorescent imaging demonstrated the uptake of the fluorescently tagged pMnO₂-NPs by most NK cells after 24 hours of exposure (FIG. 3A). Importantly, cells that had taken up the NPs showed 98% viability as assessed by Annexin V staining for apoptotic cells. This survival is similar to that observed for control NK-92 cells not exposed to the NPs (FIG. 3B).

### Example 4. pMnO₂-NP-mediated Knockdown of TGFBR2

Complexing siRNA against TGFBR2 increased gene knockdown. Gene expression analysis by qRT-PCR showed a 90% decrease in TGFBR2 mRNA levels after incubation with NP/siRNA (FIG. 3C). The siRNA alone resulted in a <10 decrease in TGFBR2 mRNA levels in the treated NK-92 cells (FIG. 3C). The qRT-PCR results were corroborated by immunofluorescence staining for the receptor. The NK-92 cells treated with siRNA only expressed TGFBR2 at levels comparable to control NK-92 cells at the protein level while NP/siRNA treated cells expressed significantly lower levels of the receptor (FIG. 3D).

### Example 5. Killing of A549 Cells by NK-92 Cells

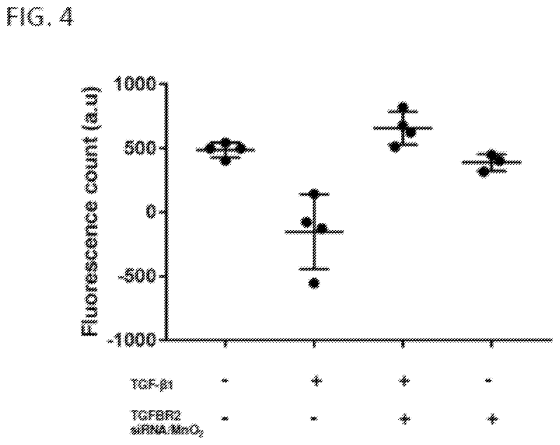

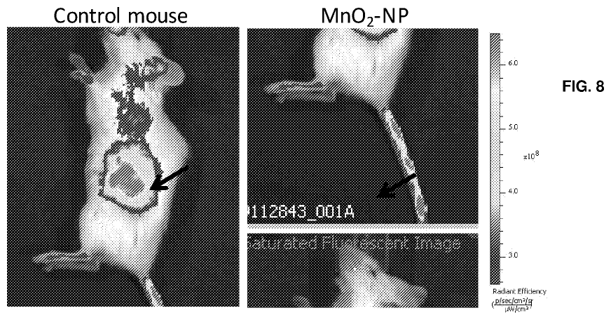

An assessment of siRNA-NP delivery to NK cells is presented in FIG. 4. NK cells were treated with TGFBR2 siRNA-NPs prior to TGF-beta 1 exposure. The results, shown in FIG. 4, demonstrated the ability to kill A549 cells (adenocarcinomic human alveolar basal epithelial cells) by RNA delivery to NK cells at levels comparable to positive control. Positive control included TGFBR2 siRNA-NPs without TGF-beta exposure (see column 4). The (+/+) group, treated with TGFBR2 siRNA-NPs and thereafter exposed to TGF-beta 1 demonstrates a significantly higher fluorescence count (p, 0.05) than the control (+/−) group without nanoparticle treatment. Also, as shown in FIG. 8, fluorescently tagged MnO2 NPs were injected intravenously into A549 tumor bearing NSG mice (left). Control mice were not injected with NPs (right). Tumor accumulation of NPs was observed after 24 hours (left).

### Example 6. TGFBR2 Knockdown Restores IFN-γ Production by NK Cells

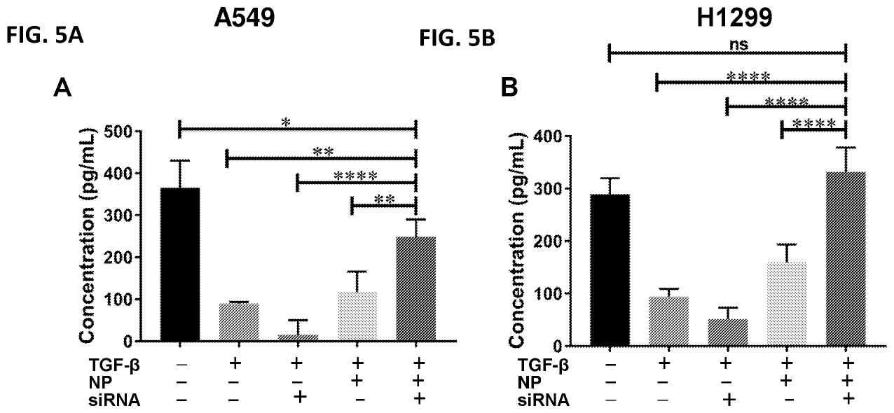

The cytokine TGF-β decreased IFN-γ production by NK-92 cells in response to both A549 and H1299 cancer cells. Treating control NK cells with TGF-β before incubation with A549 cells decreased IFN-γ production by 74%. The decrease in IFN-γ production in NK-92 cells was not restored when cells were pretreated with siRNA or NPs only. However, the treatment of NK-92 cells with siRNA complexed to NPs restored INF-γ production to 61% of that observed in control cells not treated with TGF-β (FIG. 5A).

Enhanced protection of NK-92 cells from TGF-β was observed after incubation with the metastatic H1299 cells. The TGF-β conditioned NK cells that were modified with the siRNA complexed to NPs showed similar IFN-γ production as control cells not treated with TGF-β after incubation with the cancer cells. Interestingly, there was an increase in the amount of IFN-γ produced by NK cells incubated with NPs only, though it was significantly lower than that observed with NP-siRNA treated cells (FIG. 5B).

### Example 7. NK-92 Infiltration into H1299 Spheroids

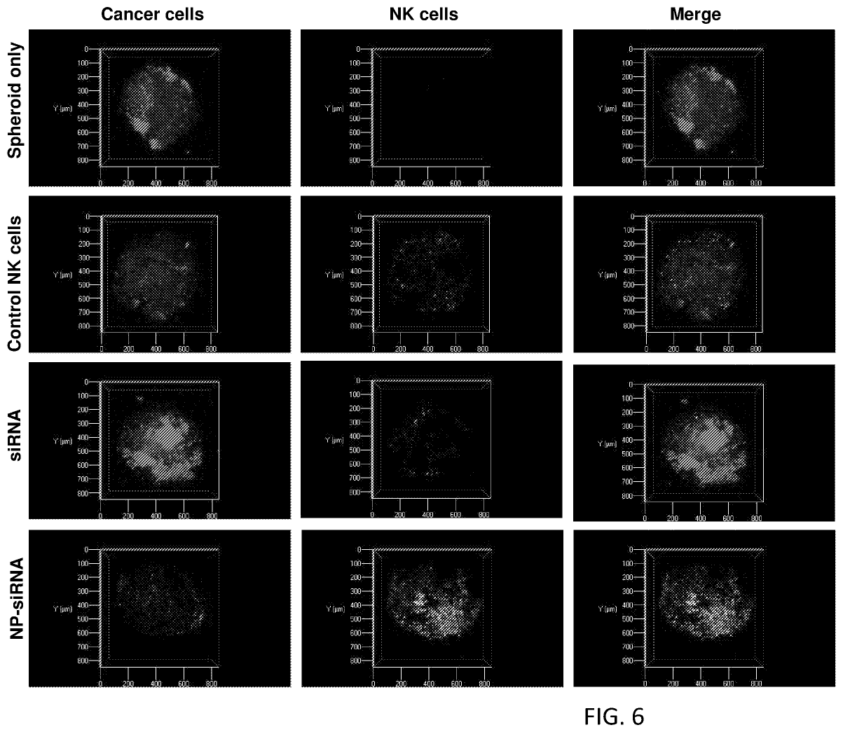

The knockdown of TGFBR2 in NK-92 cells increased their infiltration into spheroids of the metastatic H1299 cell line. Control NK-92 cells and NK-92 cells incubated with the siRNA against TGFBR2 showed minimal infiltration into the cancer spheroids. NP-mediated downregulation of the TFGBR2 protein in NK-92 cells significantly increased their interaction and infiltration into the cancer spheroids (FIG. 6).

### Sequences

Given the known sequences provided below, those skilled in the art could develop inhibitory oligonucleotides to interfere with expression thereof. Inhibitory oligonucleotides are also commercially available that target the noted sequences.

| TGF beta receptor II sequence. | |
|---|---|
| 1 | gttggcgagg agtttcctgt ttcccccgca gcgctgagtt gaagttgagt gagtcactcg |
| 61 | cgcgcacgga gcgacgacac ccccgcgcgt gcacccgctc gggacaggag ccggactcct |
| 121 | gtgcagcttc cctcggccgc cgggggcctc cccgcgcctc gccggcctcc aggcccctcc |
| 181 | tggctggcga gcgggcgcca catctggccc gcacatctgc gctgccggcc cggcgcgggg |
| 241 | tccggagagg gcgcggcgcg gagcgcagcc aggggtccgg gaaggcgccg tccgtgcgct |
| 301 | gggggctcgg tctatgacga gcagcggggt ctgccatggg tcgggggctg ctcaggggcc |
| 361 | tgtggccgct gcacatcgtc ctgtggacgc gtatcgccag cacgatccca ccgcacgttc |
| 421 | agaagtcggt taataacgac atgatagtca ctgacaacaa cggtgcagtc aagtttccac |
| 481 | aactgtgtaa attttgtgat gtgagatttt ccacctgtga caaccagaaa tcctgcatga |
| 541 | gcaactgcag catcacctcc atctgtgaga agccacagga agtctgtgtg gctgtatgga |
| 601 | gaaagaatga cgagaacata acactagaga cagtttgcca tgaccccaag ctcccctacc |
| 661 | atgactttat tctggaagat gctgcttctc caaagtgcat tatgaaggaa aaaaaaaagc |
| 721 | ctggtgagac tttcttcatg tgttcctgta gctctgatga gtgcaatgac aacatcatct |
| 781 | tctcagaaga atataacacc agcaatcctg acttgttgct agtcatattt caagtgacag |
| 841 | gcatcagcct cctgccacca ctgggagttg ccatatctgt catcatcatc ttctactgct |
| 901 | accgcgttaa ccggcagcag aagctgagtt caacctggga aaccggcaag acgcggaagc |
| 961 | tcatggagtt cagcgagcac tgtgccatca tcctggaaga tgaccgctct gacatcagct |
| 1021 | ccacgtgtgc caacaacatc aaccacaaca cagagctgct gcccattgag ctggacaccc |
| 1081 | tggtggggaa aggtcgcttt gctgaggtct ataaggccaa gctgaagcag aacacttcag |
| 1141 | agcagtttga gacagtggca gtcaagatct ttccctatga ggagtatgcc tcttggaaga |
| 1201 | cagagaagga catcttctca gacatcaatc tgaagcatga gaacatactc cagttcctga |
| 1261 | cggctgagga gcggaagacg gagttgggga aacaatactg gctgatcacc gccttccacg |
| 1321 | ccaagggcaa cctacaggag tacctgacgc ggcatgtcat cagctgggag gacctgcgca |
| 1381 | agctgggcag ctccctcgcc cgggggattg ctcacctcca cagtgatcac actccatgtg |
| 1441 | ggaggcccaa gatgcccatc gtgcacaggg acctcaagag ctccaatatc ctcgtgaaga |
| 1501 | acgacctaac ctgctgcctg tgtgactttg ggctttccct gcgtctggac cctactctgt |
| 1561 | ctgtggatga cctggctaac agtgggcagg tgggaactgc aagatacatg gctccagaag |
| 1621 | tcctagaatc caggatgaat ttggagaatg ctgagtcctt caagcagacc gatgtctact |
| 1681 | ccatggctct ggtgctctgg gaaatgacat ctcgctgtaa tgcagtggga gaagtaaaag |
| 1741 | attatgagcc tccatttggt tccaaggtgc gggagcaccc ctgtgtcgaa agcatgaagg |
| 1801 | acaacgtgtt gagagatcga gggcgaccag aaattcccag cttctggctc aaccaccagg |
| 1861 | gcatccagat ggtgtgtgag acgttgactg agtgctggga ccacgaccca gaggcccgtc |
| 1921 | tcacagccca gtgtgtggca gaacgcttca gtgagctgga gcatctggac aggctctcgg |
| 1981 | ggaggagctg ctcggaggag aagattcctg aagacggctc cctaaacact accaaatagc |
| 2041 | tcttatgggg caggctgggc atgtccaaag aggctgcccc tctcaccaaa |
| TGF beta receptor 1 | |
| 1 | aaagggccgg agcgaggccg ccgcggcggc tagggaggtg gggcgaggcg aggtttgctg |
| 61 | gggtgaggca gcggcgcggc cgggccgggc cgggccacag gcggtggcgg cgggaccatg |
| 121 | gaggcggcgg tcgctgctcc gcgtccccgg ctgctcctcc tcgtgctggc ggcggcggcg |
| 181 | gcggcggcgg cggcgctgct cccgggggcg acggcgttac agtgtttctg ccacctctgt |
| 241 | acaaaagaca attttacttg tgtgacagat gggctctgct ttgtctctgt cacagagacc |
| 301 | acagacaaag ttatacacaa cagcatgtgt atagctgaaa ttgacttaat tcctcgagat |
| 361 | aggccgtttg tatgtgcacc ctcttcaaaa actgggtctg tgactacaac atattgctgc |
| 421 | aatcaggacc attgcaataa aatagaactt ccaactactg taaagtcatc acctggcctt |
| 481 | ggtcctgtgg aactggcagc tgtcattgct ggaccagtgt gcttcgtctg catctcactc |
| 541 | atgttgatgg tctatatctg ccacaaccgc actgtcattc accatcgagt gccaaatgaa |
| 601 | gaggaccctt cattagatcg cccttttatt tcagagggta ctacgttgaa agacttaatt |
| 661 | tatgatatga caacgtcagg ttctggctca ggtttaccat tgcttgttca gagaacaatt |
| 721 | gcgagaacta ttgtgttaca agaaagcatt ggcaaaggtc gatttggaga agtttggaga |
| 781 | ggaaagtggc ggggagaaga agttgctgtt aagatattct cctctagaga agaacgttcg |
| 841 | tggttccgtg aggcagagat ttatcaaact gtaatgttac gtcatgaaaa catcctggga |
| 901 | tttatagcag cagacaataa agacaatggt acttggactc agctctggtt ggtgtcagat |
| 961 | tatcatgagc atggatccct ttttgattac ttaaacagat acacagttac tgtggaagga |
| 1021 | atgataaaac ttgctctgtc cacggcgagc ggtcttgccc atcttcacat ggagattgtt |
| 1081 | ggtacccaag gaaagccagc cattgctcat agagatttga aatcaaagaa tatcttggta |
| 1141 | aagaagaatg gaacttgctg tattgcagac ttaggactgg cagtaagaca tgattcagcc |
| 1201 | acagatacca ttgatattgc tccaaaccac agagtgggaa caaaaaggta catggcccct |
| 1261 | gaagttctcg atgattccat aaatatgaaa cattttgaat ccttcaaacg tgctgacatc |
| 1321 | tatgcaatgg gcttagtatt ctgggaaatt gctcgacgat gttccattgg tggaattcat |
| 1381 | gaagattacc aactgcctta ttatgatctt gtaccttctg acccatcagt tgaagaaatg |
| 1441 | agaaaagttg tttgtgaaca gaagttaagg ccaaatatcc caaacagatg gcagagctgt |
| 1501 | gaagccttga gagtaatggc taaaattatg agagaatgtt ggtatgccaa tggagcagct |
| 1561 | aggcttacag cattgcggat taagaaaaca ttatcgcaac tcagtcaaca ggaaggcatc |
| 1621 | aaaatgtaat tctacagctt tgcctgaact ctcctttttt cttcagatct gctcctgggt |
| 1681 | tttaatttgg gaggtcaatt gttctacctc actgagaggg aacagaagga tattgcttcc |
| 1741 | ttttgcagca gtgtaataaa gtcaattaaa aacttcccag gatttctttg gacccaggaa |
| 1801 | acagccatgt gggtcctttc tgtgcactat gaacgcttct ttcccaggac agaaaatgtg |
| 1861 | tagtctacct ttatttttta ttaacaaaac ttgtttttta aaaagatgat tgctggtctt |
| 1921 | aactttaggt aactctgctg tgctggagat catctttaag ggcaaaggag ttggattgct |
| 1981 | gaattacaat gaaacatgtc ttattactaa agaaagtgat ttactcctgg ttagtacatt |
| 2041 | ctcagaggat tctgaaccac tagagtttcc ttgattcaga ctttgaatgt actgttctat |
| 2101 | agtttttcag gatcttaaaa ctaacactta taaaactctt atcttgagtc taaaaatgac |
| 2161 | ctcatatagt agtgaggaac ataattcatg caattgtatt ttgtatacta ttattgttct |
| 2221 | ttcacttatt cagaacatta catgccttca aaatgggatt gtactatacc agtaagtgcc |
| 2281 | acttctgtgt ctttctaatg gaaatgagta gaattgctga aagtctctat gttaaaacct |
| 2341 | atagtgtttg aattcaaaaa gcttatttat ctgggtaacc caaacttttt ctgttttgtt |
| 2401 | tttggaaggg tttttgtggt atgtcatttg gtattctatt ctgaaaatgc ctttctccta |
| 2461 | ccaaaatgtg cttaagccac taaagaaatg aagtggcatt aattagtaaa ttattagcat |
| 2521 | ggtcatgttt gaatattctc acatcaagct tttgcatttt aattgtgttg tctaagtata |
| 2581 | cttttaaaaa atcaagtggc actctagatg cttatagtac tttaatattt gtagcataca |
| 2641 | gactaatttt tctaaaaggg aaagtctgtc tagctgcttg tgaaaagtta tgtggtattc |
| 2701 | tgtaagccat ttttttcttt atctgttcaa agacttattt tttaagacat gaattacatt |
| 2761 | taaaattaga atatggttaa tattaaataa taggcctttt tctaggaagg cgaaggtagt |
| 2821 | taataatttg aatagataac agatgtgcaa gaaagtcaca tttgttatgt atgtaggagt |
| 2881 | aaacgttcgg tggatcctct gtctttgtaa ctgaggttag agctagtgtg gttttgaggt |
| 2941 | ctcactacac tttgaggaag gcagctttta attcagtgtt tccttatgtg tgcgtacatt |
| 3001 | gcaactgctt acatgtaatt tatgtaatgc attcagtgca cccttgttac ttgggagagg |
| 3061 | tggtagctaa agaacattct gagtataggt ttttctccat ttacagatgt ctttggtcaa |
| 3121 | atattgaaag caaacttgtc atggtcttct tacattaagt tgaaactagc ttataataac |
| 3181 | tggtttttac ttccaatgct atgaagtctc tgcagggctt ttacagtttt cgaagtcctt |
| 3241 | ttatcactgt gatcttattc tgaggggaga aaaaactatc atagctctga ggcaagactt |
| 3301 | cgactttata gtgctatcag ttccccgata cagggtcaga gtaacccata cagtattttg |
| 3361 | gtcaggaaga gaaagtggcc atttacactg aatgagttgc attctgataa tgtcttatct |
| 3421 | cttatacgta gaataaattt gaaagactat ttgatcttaa aaccaaagta attttagaat |
| 3481 | gagtgacata ttacatagga atttagtgtc aatttcatgt gtttaaaaac atcatgggaa |
| 3541 | aaatgcttag aggttactat tttgactaca aagttgagtt tttttctgta gttaccataa |
| 3601 | tttcattgaa gcaaatgaat gagtttgaga ggtttgtttt tatagttgtg ttgtattact |
| 3661 | tgtttaataa taatctctaa ttctgtgatc aggtactttt tttgtggggg tttttttttt |
| 3721 | gttttttttt ttttgttgtt gtttttgggc catttctaag cctaccagat ctgctttatg |
| 3781 | aaatccaggg gaccaatgca ttttatcact aaaactattt ttatataatt ttaagaatat |
| 3841 | accaaaagtt gtctgattta aagttgtaat acatgatttc tcactttcat gtaaggttat |
| 3901 | ccacttttgc tgaagatatt ttttattgaa tcaaagattg agttacaatt atacttttct |
| 3961 | tacctaagtg gataaaatgt acttttgatg aatcagggaa tttttttaaa gttggagttt |
| 4021 | agttctaaat tgactttacg tattactgca gttaattcct tttttggcta gggatggttt |
| 4081 | gataaaccac aattggctga tattgaaaat gaaagaaact taaaaggtgg gatggatcat |
| 4141 | gattactgtc gataactgca gataaatttg attagagtaa taattttgtc atttaaaaac |
| 4201 | acagttgttt atactgccca tcctaggatg ctcaccttcc aagattcaac gtggctaaaa |
| 4261 | catcttctgg taaattgtgc gtccatattc attttgtcag tagccaggag aaatggggat |
| 4321 | gggggaaata cgacttagtg aggcatagac atccctggtc catcctttct gtctccagct |
| 4381 | gtttcttgga acctgctctc ctgcttgctg gtccctgacg cagagaccgt tgcctccccc |
| 4441 | acagccgttt gactgaaggc tgctctggag acctagagta aaacggctga tggaagttgt |
| 4501 | gggacccact tccatttcct tcagtcatta gaggtggaag ggaggggtct ccaagtttgg |
| 4561 | agattgagca gatgaggctt gggatgcccc tgctttgact tcagccatgg atgaggagtg |
| 4621 | ggatggcagc aaggtggctc ctgtggcagt ggagttgtgc cagaaacagt ggccagttgt |
| 4681 | atcgcctata agacagggta aggtctgaag agctgagcct gtaattctgc tgtaataatg |
| 4741 | atagtgctca agaagtgcct tgagttggtg tacagtgcca tggccatcaa gaatcccaga |
| 4801 | tttcaggttt tattacaaaa tgtaagtggt cacttggcga ttttgtagta catgcatgag |
| 4861 | ttaccttttt tctctatgtc tgagaactgt cagattaaaa caagatggca aagagatcgt |
| 4921 | tagagtgcac aacaaaatca ctatcccatt agacacatca tcaaaagctt atttttattc |
| 4981 | ttgcactgga agaatcgtaa gtcaactgtt tcttgaccat ggcagtgttc tggctccaaa |
| 5041 | tggtagtgat tccaaataat ggttctgtta acactttggc agaaaatgcc agctcagata |
| 5101 | ttttgagata ctaaggatta tctttggaca tgtactgcag cttcttgtct ctgttttgga |
| 5161 | ttactggaat acccatgggc cctctcaaga gtgctggact tctaggacat taagatgatt |
| 5221 | gtcagtacat taaacttttc aatcccatta tgcaatcttg tttgtaaatg taaacttcta |
| 5281 | aaaatatggt taataacatt caacctgttt attacaactt aaaaggaact tcagtgaatt |
| 5341 | tgtttttatt ttttaacaag atttgtgaac tgaatatcat gaaccatgtt ttgatacccc |
| 5401 | tttttcacgt tgtgccaacg gaatagggtg tttgatattt cttcatatgt taaggagatg |
| 5461 | cttcaaaatg tcaattgctt taaacttaaa ttacctctca agagaccaag gtacatttac |
| 5521 | ctcattgtgt atataatgtt taatatttgt cagagcattc tccaggtttg cagttttatt |
| 5581 | tctataaagt atgggtatta tgttgctcag ttactcaaat ggtactgtat tgtttatatt |
| 5641 | tgtaccccaa ataacatcgt ctgtactttc tgttttctgt attgtatttg tgcaggattc |
| 5701 | tttaggcttt atcagtgtaa tctctgcctt ttaagatatg tacagaaaat gtccatataa |
| 5761 | atttccattg aagtcgaatg atactgagaa gcctgtaaag aggagaaaaa aacataagct |
| 5821 | gtgtttcccc ataagttttt ttaaattgta tattgtattt gtagtaatat tccaaaagaa |
| 5881 | tgtaaatagg aaatagaaga gtgatgctta tgttaagtcc taacactaca gtagaagaat |
| 5941 | ggaagcagtg caaataaatt acatttttcc caagtgccag tggcatattt taaaataaag |
| 6001 | tgtatacgtt ggaatgagtc atgccatatg tagttgctgt agatggcaac tagaaccttt |
| 6061 | gagttacaag agtctttaga agttttctaa ccctgcctag tgcaagttac aatattatag |
| 6121 | cgtgttcggg gagtgccctc ctgtctgcag gtgtgtctct gtgcctgggg gcttttctcc |
| 6181 | acatgcttag gggtgtgggt cttccattgg ggcatgatgg acctgtctac aggtgatctc |
| 6241 | tgttgccttt gggtcagcac atttgttagt ctcctggggg tgaaaacttg gcttacaaga |
| 6301 | gaactggaaa aatgatgaga tgtggtcccc aaacccttga ttgactctgg ggaggggctt |
| 6361 | tgtgaatagg attgctctca cattaaagat agttacttca atttgaaggc tggatttagg |
| 6421 | gatttttttt tttccttata acaaagacat caccaggata tgaagctttt gttgaaagtt |
| 6481 | ggaaaaaaag tgaaattaaa gacattccca gacaaa |
| -J Invest Dermatol. 2014 July; 134(7): 2016-2025 | |
| TGF-beta 1 | |
| 1 | acctccctcc gcggagcagc cagacagcga gggccccggc cgggggcagg ggggacgccc |
| 61 | cgtccggggc acccccccgg ctctgagccg cccgcggggc cggcctcggc ccggagcgga |
| 121 | ggaaggagtc gccgaggagc agcctgaggc cccagagtct gagacgagcc gccgccgccc |
| 181 | ccgccactgc ggggaggagg gggaggagga gcgggaggag ggacgagctg gtcgggagaa |
| 241 | gaggaaaaaa acttttgaga cttttccgtt gccgctggga gccggaggcg cggggacctc |
| 301 | ttggcgcgac gctgccccgc gaggaggcag gacttgggga ccccagaccg cctccctttg |
| 361 | ccgccgggga cgcttgctcc ctccctgccc cctacacggc gtccctcagg cgcccccatt |
| 421 | ccggaccagc cctcgggagt cgccgacccg gcctcccgca aagacttttc cccagacctc |
| 481 | gggcgcaccc cctgcacgcc gccttcatcc ccggcctgtc tcctgagccc ccgcgcatcc |
| 541 | tagacccttt ctcctccagg agacggatct ctctccgacc tgccacagat cccctattca |
| 601 | agaccaccca ccttctggta ccagatcgcg cccatctagg ttatttccgt gggatactga |
| 661 | gacacccccg gtccaagcct cccctccacc actgcgccct tctccctgag gacctcagct |
| 721 | ttccctcgag gccctcctac cttttgccgg gagaccccca gcccctgcag gggcggggcc |
| 781 | tccccaccac accagccctg ttcgcgctct cggcagtgcc ggggggcgcc gcctccccca |
| 841 | tgccgccctc cgggctgcgg ctgctgccgc tgctgctacc gctgctgtgg ctactggtgc |
| 901 | tgacgcctgg ccggccggcc gcgggactat ccacctgcaa gactatcgac atggagctgg |
| 961 | tgaagcggaa gcgcatcgag gccatccgcg gccagatcct gtccaagctg cggctcgcca |
| 1021 | gccccccgag ccagggggag gtgccgcccg gcccgctgcc cgaggccgtg ctcgccctgt |
| 1081 | acaacagcac ccgcgaccgg gtggccgggg agagtgcaga accggagccc gagcctgagg |
| 1141 | ccgactacta cgccaaggag gtcacccgcg tgctaatggt ggaaacccac aacgaaatct |
| 1201 | atgacaagtt caagcagagt acacacagca tatatatgtt cttcaacaca tcagagctcc |
| 1261 | gagaagcggt acctgaaccc gtgttgctct cccgggcaga gctgcgtctg ctgaggctca |
| 1321 | agttaaaagt ggagcagcac gtggagctgt accagaaata cagcaacaat tcctggcgat |
| 1381 | acctcagcaa ccggctgctg gcacccagcg actcgccaga gtggttatct tttgatgtca |
| 1441 | ccggagttgt gcggcagtgg ttgagccgtg gaggggaaat tgagggcttt cgccttagcg |
| 1501 | cccactgctc ctgtgacagc agggataaca cactgcaagt ggacatcaac gggttcacta |
| 1561 | ccggccgccg aggtgacctg gccaccattc atggcatgaa ccggcctttc ctgcttctca |
| 1621 | tggccacccc gctggagagg gcccagcatc tgcaaagctc ccggcaccgc cgagccctgg |
| 1681 | acaccaacta ttgcttcagc tccacggaga agaactgctg cgtgcggcag ctgtacattg |
| 1741 | acttccgcaa ggacctcggc tggaagtgga tccacgagcc caagggctac catgccaact |
| 1801 | tctgcctcgg gccctgcccc tacatttgga gcctggacac gcagtacagc aaggtcctgg |
| 1861 | ccctgtacaa ccagcataac ccgggcgcct cggcggcgcc gtgctgcgtg ccgcaggcgc |
| 1921 | tggagccgct gcccatcgtg tactacgtgg gccgcaagcc caaggtggag cagctgtcca |
| 1981 | acatgatcgt gcgctcctgc aagtgcagct gaggtcccgc cccgccccgc cccgccccgg |
| 2041 | caggcccggc cccaccccgc cccgcccccg ctgccttgcc catgggggct gtatttaagg |
| 2101 | acacccgtgc cccaagccca cctggggccc cattaaagat ggagagagga ctgcggatct |
| 2161 | ctgtgtcatt gggcgcctgc ctggggtctc catccctgac gttcccccac tcccactccc |
| 2221 | tctctctccc tctctgcctc ctcctgcctg tctgcactat tcctttgccc ggcatcaagg |
| 2281 | cacaggggac cagtggggaa cactactgta gttagatcta tttattgagc accttgggca |
| 2341 | ctgttgaagt gccttacatt aatgaactca ttcagtcacc atagcaacac tctgagatgc |
| 2401 | agggactctg ataacaccca ttttaaaggt gaggaaacaa gcccagagag gttaagggag |
| 2461 | gagttcctgc ccaccaggaa cctgctttag tgggggatag tgaagaagac aataaaagat |
| 2521 | agtagttcag gccaggcggg gtggctcacg cctgtaatcc tagcactttt gggaggcaga |
| 2581 | gatgggagga ttacttgaat ccaggcattt gagaccagcc tgggtaacat agtgagaccc |
| 2641 | tatctctaca aaacactttt aaaaaatgta cacctgtggt cccagctact ctggaggcta |
| 2701 | aggtgggagg atcacttgat cctgggaggt caaggctgca g |

-Gene Therapy volume 22, pages 333-340 (2015) (provides example of
TGFbeta siRNA).

The above studies were supported by a grant from the State of Florida Department of Health and James & Esther King Biomedical Research Program subaward #30-18456-99 under Prime Award #6JK01.

### REFERENCES

References listed throughout the specification are hereby incorporated by reference in their entirety.

What is claimed is:

1. A method of inducing an anti-cancer immune response in a subject, comprising administering to immune cells of the subject in vivo, or to immune cells to be delivered to the subject, ex vivo, a pharmaceutically effective amount of a nanoparticle composition comprising a manganese dioxide nanoparticle (MnO₂-NP) complexed with a nucleic acid sequence in an amount sufficient to induce, enhance, or promote an immune response against the cancer in the subject.

2. The method of claim 1, wherein the immune cells comprise natural killer cells (NK cells).

3. The method of claim 1, wherein the nucleic acid sequence comprises an RNA sequence.

4. The method of claim 3, wherein the RNA sequence comprises an inhibitory oligonucleotide for silencing transforming growth factor beta (TGF-β), TGF-βrI or TGF-βrII.

5. The method of claim 4, wherein the RNA sequence comprises a miRNA, siRNA, or shRNA sequence.

6. The method of claim 1, wherein the nanoparticle composition is administered to the immune cells ex vivo by: 1) obtaining immune cells to be treated from a subject or from an allogeneic source; 2) treating the immune cells with the nanoparticle composition; and 3) introducing the immune cells into the subject to activate NK cells to induce, enhance, or promote an immune response against the cancer in the subject.

7. The method of claim 1, wherein the nanoparticle composition is administered to the immune cells in vivo.

8. A nanoparticle composition, which is produced by:
(a) combining poly (allylamine hydrochloride) (PAH) and KMnO4 in a 1:1 ratio to water with mixing;
(b) washing the nanoparticles formed and suspending the nanoparticles in water;
(c) adding succinimidyl valerate poly(ethylene glycol) to the nanoparticles, with further mixing;
(d) washing the nanoparticles; and
(e) complexing a silencing nucleic acid sequence to the nanoparticles to form a manganese dioxide nanoparticle (MnO₂-NP) complexed with a nucleic acid sequence.

9. The nanoparticle composition of claim 8, wherein the nanoparticles have a size of less than 150 nm.

10. The nanoparticle composition of claim 9, wherein the nanoparticles have a size of less than 100 nm.

11. A nanoparticle formulation, comprising the nanoparticle composition of claim 8 and a pharmaceutically acceptable carrier.

12. A nanoparticle formulation, comprising:
(a) a plurality of manganese dioxide nanoparticles conjugated to a nucleic acid sequence for silencing TGF-β, said nanoparticles having a size by transmission electron microscopy of less than 100 nm;
(b) a stabilizer comprising succinimidyl valerate poly(ethylene glycol); and
(c) a pharmaceutically acceptable carrier.

13. The nanoparticle formulation of claim 12, wherein the nucleic acid sequence comprises a miRNA, siRNA, or shRNA.

14. A method of treating cancer in a subject in need thereof, comprising administering to the subject the nanoparticle formulation of claim 12.

15. A method of treating cancer in a subject in need thereof, comprising administering to the subject or to immune cells to be delivered to the subject, the nanoparticle composition of claim 8.

16. The method of claim 15, wherein administering comprises ex vivo administration of nanoparticle composition to immune cells comprising:
a) obtaining immune cells to be treated from a subject, or from an allogeneic source; b) treatment of the immune cells with the nanoparticle composition; and c) introduction of the immune cells into the subject to activate NK cells to induce, enhance, or promote an immune response against the cancer in the subject.


SEQUENCE LISTING (7 sequences)

SEQ ID NO: 1 - DNA, 19 bp, Artificial Sequence
  TGFBR2 primer
acgtgttgagagatcgagg

SEQ ID NO: 2 - DNA, 20 bp, Artificial Sequence
  TGFBR2 primer
cccagcactcagtcaacgtc

SEQ ID NO: 3 - DNA, 20 bp, Artificial Sequence
  GAPDH primer
tagacgggaagctcactggc

SEQ ID NO: 4 - DNA, 20 bp, Artificial Sequence
  GAPDH primer
aggtccaccaccctgttgct

SEQ ID NO: 5 - DNA, 2090 bp, Homo sapiens
gttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggcccctcctggctggcgagcgggcgccacatctggcccgcacatctgcgctgccggcccggcgcggggtccggagagggcgcggcgcggagcgcagccaggggtccgggaaggcgccgtccgtgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgctgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttatggggcaggctgggcatgtccaaagaggctgcccctctcaccaaa

SEQ ID NO: 6 - DNA, 6516 bp, Homo sapiens
aaagggccggagcgaggccgccgcggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatctctgccttttaagatatgtacagaaaatgtccatataaatttccattgaagtcgaatgatactgagaagcctgtaaagaggagaaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaagtgccagtggcatattttaaaataaagtgtatacgttggaatgagtcatgccatatgtagttgctgtagatggcaactagaacctttgagttacaagagtctttagaagttttctaaccctgcctagtgcaagttacaatattatagcgtgttcggggagtgccctcctgtctgcaggtgtgtctctgtgcctgggggcttttctccacatgcttaggggtgtgggtcttccattggggcatgatggacctgtctacaggtgatctctgttgcctttgggtcagcacatttgttagtctcctgggggtgaaaacttggcttacaagagaactggaaaaatgatgagatgtggtccccaaacccttgattgactctggggaggggctttgtgaataggattgctctcacattaaagatagttacttcaatttgaaggctggatttagggatttttttttttccttataacaaagacatcaccaggatatgaagcttttgttgaaagttggaaaaaaagtgaaattaaagacattcccagacaaa

SEQ ID NO: 7 - DNA, 2741 bp, Homo sapiens
acctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggccaggcggggtggctcacgcctgtaatcctagcacttttgggaggcagagatgggaggattacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag